(19) United States
(12) Patent Application Publication
(10) Pub. No.: US 2009/0148849 A1
(43) Pub. Date: Jun. 11, 2009

(54) ONE-STEP TARGET DETECTION ASSAY

(76) Inventors: Barbara Galvan-Goldman, Etobicoke (CA); Agnes Zoldowski, Aurora (CA); Susan Bortolin, Oakville (CA); Frank Merante, Etobicoke (CA)

Correspondence Address: FULBRIGHT & JAWORSKI L.L.P., 600 CONGRESS AVE., SUITE 2400, AUSTIN, TX, 78701, US

(21) Appl. No.: 12/262,842

(22) Filed: Oct. 31, 2008

Related U.S. Application Data
(60) Provisional application No. 60/984,982, filed on Nov. 2, 2007.

(51) Int. Cl. C12Q 1/68; C12P 19/34

(52) U.S. Cl. 435/6; 435/91.2

(57) ABSTRACT

The present invention provides nucleic acid amplification, detection, and genotyping techniques. In one embodiment, the present invention provides a method for amplifying and detecting a target nucleic acid sequence by providing a first primer pair comprising: a first primer comprising a target specific sequence, a tag sequence 5′ of the target specific sequence, and a blocker between the target specific sequence and the tag sequence, and a second primer comprising a target specific sequence; providing a reporter attached to either the second primer or to a dNTP; providing a capture complex comprising an anti-tag sequence attached to a solid support; combining the first primer pair, the capture complex, the reporter, and a sample comprising a target nucleic acid sequence under conditions suitable for amplification of the target nucleic acid sequence and hybridization of the amplified target nucleic acid sequence to the capture complex; and detecting the amplified target nucleic acid sequence.

This application claims priority to U.S. Provisional Patent Application No. 60/984,982, filed on Nov. 2, 2007, which is incorporated herein by reference.

### BACKGROUND OF THE INVENTION

1. Field of the Invention

The present invention relates generally to the fields of genetics and molecular biology. More particularly, it concerns genetic sequence detection. In specific embodiments, the invention concerns methods for screening for mutations and polymorphisms and methods for detecting and typing pathogens by analysis of their DNA or RNA.

2. Description of Related Art

Nucleic acid amplification and detection techniques are frequently employed in analyzing DNA samples for mutations and polymorphisms. They are also employed in the detection and typing of infectious pathogens by analysis of their DNA or RNA. Approaches such as allele-specific PCR (AS-PCR) and allele-specific primer extension (ASPE) detect mutations and polymorphisms using oligonucleotide primers selected such that they selectively achieve primer extension of either a sequence containing a variant nucleotide or the corresponding sequence containing the wild-type nucleotide. For example, U.S. Pat. No. 5,595,890 describes AS-PCR and ASPE methods in which the diagnostic nucleotide (i.e., the nucleotide that is selected to be complementary to either the wild-type nucleotide or a variant nucleotide at a given position in the target sequence) is at the 3′ terminal end of the primer. When the terminal nucleotide of the primer is complementary to the corresponding nucleotide in the target sequence an extension product is synthesized. When the terminal nucleotide of the primer is not complementary to the corresponding nucleotide in the target sequence no extension product is synthesized.

U.S. Pat. No. 5,639,611 describes an AS-PCR assay in which the 3′-terminal nucleotide of one of the primers of a primer set forms a match with one allele and a mismatch with the other allele of a target DNA. U.S. Pat. No. 5,639,611 also describes that one primer may be biotin labeled and the other primer fluorescently labeled for capture and detection. U.S. Pat. No. 5,137,806 also describes AS-PCR and ASPE using a diagnostic primer in which the diagnostic nucleotide is at the 3′ terminus. U.S. Pat. No. 5,137,806 also contemplates including mismatch nucleotides between the target sequence complementary region and the template binding region in order to increase the “3′ terminal flap” that occurs when the target sequence is not present. The disclosures of U.S. Pat. Nos. 5,595,890, 5,639,611, and 5,137,806 are incorporated by reference.

Despite the usefulness of the above-mentioned techniques, better methods of nucleic acid amplification and detection that can provide quicker results with less risk of error are needed.

### SUMMARY OF THE INVENTION

The methods and compositions of the present invention provide nucleic acid amplification, detection, and genotyping techniques. In one embodiment, the present invention provides a method for amplifying a target nucleic acid sequence comprising: providing a first primer pair comprising: a first primer comprising a target specific sequence, a tag sequence 5′ of the target specific sequence, and a blocker between the target specific sequence and the tag sequence, a second primer comprising a target specific sequence; a reporter attached to either the second primer or to a dNTP; providing a capture complex comprising an anti-tag sequence attached to a solid support; and combining the first primer pair, the reporter, the capture complex, and a sample comprising a target nucleic acid sequence under conditions suitable for amplification of the target nucleic acid sequence and hybridization of the amplified target nucleic acid sequence to the capture complex. In another embodiment, the present invention provides a method for amplifying a target nucleic acid sequence comprising: providing a first primer pair comprising: a first primer comprising a target specific sequence, a tag sequence 5′ of the target specific sequence, and a blocker between the target specific sequence and the tag sequence, a second primer comprising a target specific sequence and a reporter; providing a capture complex comprising an anti-tag sequence attached to a solid support; and combining the first primer pair, the capture complex, and a sample comprising a target nucleic acid sequence under conditions suitable for amplification of the target nucleic acid sequence and hybridization of the amplified target nucleic acid sequence to the capture complex.

The target nucleic acid sequence may be any sequence of interest. The methods of the present invention are well-suited for screening DNA or RNA samples for mutations and polymorphisms or for detecting large deletions. For example, the methods of the present invention may be used in genetic screening for disease such as cystic fibrosis, Tay-Sachs disease, Canavan disease, familial dysautomonia, Gaucher disease, Bloom's syndrome, Fanconi anemia GrpC, Nieman-Pick, and Mucolipidosis type IV. The present invention may also be used in genetic screening for mutations and/or polymorphisms associated with defects in coagulation or drug metabolism. In addition, the methods of the present invention may be used in the detection and typing of infectious pathogens (e.g., viruses and bacteria) by detection and analysis of their DNA or RNA. The sample containing the target nucleic acid sequence may be any sample that contains nucleic acids. In certain aspects of the invention the sample is, for example, a subject who is being screened for the presence or absence of one or more genetic mutations or polymorphisms. In another aspect of the invention the sample may be from a subject who is being tested for the presence or absence of a pathogen. Where the sample is obtained from a subject, it may be obtained by methods known to those in the art such as aspiration, biopsy, swabbing, venipuncture, spinal tap, fecal sample, or urine sample. In certain embodiments the subject is a mammal, bird, or fish. The mammal may be, for example, a human, cat, dog, cow, horse, sheep, swine, swine, rabbit, rat, or mouse. In some aspects of the invention, the sample is an environmental sample such as a water, soil, or air sample. In other aspects of the invention, the sample is from a plant, bacteria, virus, fungi, protozoan, or metazoan.

The primer pairs according to one aspect of the invention comprise a first primer comprising a target specific sequence, a tag sequence 5′ of the target specific sequence, and a blocker between the target specific sequence and the tag sequence, and a second primer comprising a target specific sequence. The second primer may further comprise a reporter. In other embodiments, the reporter is attached to a dNTP, which results in the reporter being incorporated into the amplified nucleic acid sequence. The primers are designed to amplify the target nucleic acid sequence. Accordingly, each of the primers contains a target specific sequence, which selectively hybridizes to a sequence on the target. To detect mutations and polymorphisms by AS-PCR or ASPE one or both of the primers of the primer pair may be selected such that it selectively achieves primer extension of either a sequence containing a variant nucleotide or the corresponding sequence containing the wild-type nucleotide.

A tag sequence is located 5′ of the target specific sequence on one of the two primers in a primer pair. Amplification of the target nucleic acid sequence with the primer pair will, therefore, result in one strand of a double stranded amplification product being tagged with the tag sequence. The tag sequence is the complement of the anti-tag sequence of the capture complex. Specific hybridization between the tag sequence and the anti-tag sequence allows for the capture of the amplified target nucleic acid sequence by the capture complex. In certain embodiments, the tag sequence is between 6 to 60, 8 to 50, 10 to 40, or 20 to 30 nucleotides in length. In one embodiment, the tag sequence is 24 nucleotides in length. A number of tag and tag complement sequences are known in the art and may be used in the present invention. For example, U.S. Pat. No. 7,226,737, incorporated herein by reference, describes a set of 210 non-cross hybridizing tags and anti-tags. In addition, U.S. Published Application No. 2005/0191625, incorporated herein by reference, discloses a family of 1168 tag sequences with a demonstrated ability to correctly hybridize to their complementary sequences with minimal cross hybridization.

Following the initial extension of the primer comprising the tag sequence, the tagged extension product may serve as a template for the other primer of the primer pair. It would be undesirable, however, for the extension from such a template to proceed through the tag region as this would interfere with the hybridization of the tag sequence with the anti-tag sequence of the capture complex. Accordingly, a blocker is positioned between the target specific sequence and the tag sequence of the primer. The blocker moiety inhibits extension through the tag sequence during second strand synthesis. Non-limiting examples of blocker moieties include C6-20 straight chain alkylenes, iSp18 (which is an 18-atom hexa-ethyleneglycol), and an oligonucleotide sequence in the reverse orientation as compared to the target specific sequence. Where the blocker moiety is an oligonucleotide sequence in the reverse orientation as compared to the target specific sequence, the blocker moiety and the tag sequence may be the same—in other words, the tag sequence is placed in the opposite orientation to the target specific sequence, which inhibits polymerase extension into the tag sequence.

In some embodiments, at least one primer of a primer pair is labeled with a reporter. In some embodiments, neither primer of a primer pair is labeled with a reporter. In embodiments in which neither primer of a primer pair is labeled with a reporter, the reporter is typically attached to a dNTP present in the amplification reaction. A reporter is a molecule that facilitates the detection of a molecule to which it is attached. In a preferred embodiment, the reporter is attached to the primer that does not include a tag sequence. Numerous reporter molecules that may be used to label nucleic acids are known. Direct reporter molecules include fluorophores, chromophores, and radiophores. Non-limiting examples of fluorophores include, a red fluorescent squarine dye such as 2,4-Bis[1,3,3-trimethyl-2-indolinylidenemethyl]cyclobutenediylium-1,3-dioxolate, an infrared dye such as 2,4 Bis[3,3-dimethyl-2-(1H-benz[e]indolinylidenemethyl)]cyclobutenediylium-1,3-dioxolate, or an orange fluorescent squarine dye such as 2,4-Bis[3,5-dimethyl-2-pyrrolyl]cyclobutenediylium-1,3-diololate. Additional non-limiting examples of fluorophores include quantum dots, Alexa Fluor® dyes, AMCA, BODIPY® 630/650, BODIPY® 650/665, BODIPY®-FL, BODIPY®—R6G, BODIPY®-TMR, BODIPY®-TRX, Cascade Blue®, CyDye™, including but not limited to Cy2™, Cy3™, and Cy5™, a DNA intercalating dye, 6-FAM™, Fluorescein, HEX™, 6-JOE, Oregon Green® 488, Oregon Green® 500, Oregon Green® 514, Pacific Blue™, REG, phycobilliproteins including, but not limited to, phycoerythrin and allophycocyanin, Rhodamine Greem™, Rhodamine Red™, ROX™, TAMRA™, TET™, Tetramethylrhodamine, or Texas Red®. A signal amplification reagent, such as tyramide (PerkinElmer), may be used to enhance the fluorescence signal. Indirect reporter molecules include biotin, which must be bound to another molecule such as streptavidin-phycoerythrin for detection. In a multiplex reaction, the reporter attached to the primer or the dNTP may be the same for all reactions in the multiplex reaction because the identities of the amplification products may be determined based on the identity of the solid support to which they hybridize as discussed in more detail below.

As mentioned above, a tag sequence is located 5′ of the target specific sequence on one of the two primers in a primer pair. The tag sequence is the complement of the anti-tag sequence of the capture complex. Specific hybridization between the tag sequence and the anti-tag sequence allows for the capture of the amplified target nucleic acid sequence by the capture complex. In certain embodiments, the anti-tag sequence is between 6 to 60, 8 to 50, 10 to 40, or 20 to 30 nucleotides in length. In one embodiment, the anti-tag sequence is 24 nucleotides in length. As mentioned above, a number of tag and tag complement sequences are known in the art and may be used in the present invention. For example, U.S. Pat. No. 7,226,737 describes a set of 210 non-cross hybridizing tags and anti-tags. In addition, U.S. Published Application No. 2005/0191625 discloses a family of 1168 tag sequences with a demonstrated ability to correctly hybridize to their complementary sequences with minimal cross hybridization.

The capture complex also comprises a solid support. Non-limiting examples of solid supports include: nitrocellulose, nylon membrane, glass, activated quartz, activated glass, polyvinylidene difluoride (PVDF) membrane, polystyrene substrates, polyacrylamide-based substrate, other polymers, copolymers, or crosslinked polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules). A solid support may be in the form of, for example, a bead (microsphere), a column, or a chip.

The beads of the capture complex may be encoded such that one subpopulation of beads can be distinguished from another subpopulation. Encoding may be by a variety of techniques. For example, the beads may be fluorescently labeled with fluorescent dyes having different emission spectra and/or different signal intensities. In certain embodiments, the beads are Luminex FlexMAP™ microspheres or Luminex xMAP® microspheres. The size of the beads in a subpopulation may also be used to distinguish one subpopulation from another. Another method of modifying a bead is to incorporate a magnetically responsive substance, such as Fe₃O₄, into the structure. Paramagnetic and superparamagnetic microspheres have negligible magnetism in the absence of a magnetic field, but application of a magnetic field induces alignment of the magnetic domains in the microspheres, resulting in attraction of the microspheres to the field source. Combining fluorescent dyes, bead size, and/or magnetically responsive substances into the beads can further increase the number of different subpopulations of beads that can be created.

In one embodiment, the method further comprises detecting the amplified target nucleic acid sequence. Detection of the amplified target nucleic acid may be by a variety of techniques. In one aspect of the invention, the amplified target nucleic acids are detected using a flow cytometer. Flow cytometry is particularly well-suited where the solid support of the capture complex is a bead or other particle. In other aspects of the invention, detecting the amplified target nucleic acid comprises imaging the amplified target nucleic acid sequence bound to the capture complex. The imaging may be on, for example, a bead array platform or a chip array platform.

The methods of the present invention may be used in multiplexed assays. In such multiplexed assay, the sample will typically comprise at least a second target nucleic acid sequence. In certain aspects of the invention, there are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 120, 140, 160, 180, 200, 220, 240, 260, 280, 300, 400, 500, 600, 700, 800, 900, 1000, or any range derivable therein, target nucleic acid sequences in the sample. As mentioned above, a target nucleic acid sequence may be any sequence of interest. One target nucleic acid sequence may be in the same gene or a different gene as another target nucleic acid sequence, and the target nucleic acid sequences may or may not overlap. Of course, a target nucleic acid sequence need not be within a gene but may be within, for example, a non-coding region of DNA. In a multiplex assay where at least a second target nucleic acid to be amplified is present in a sample, at least a second discriminating primer or a second primer pair is combined with the first primer pair, the capture complex, and the sample comprising the target nucleic acid sequences under conditions suitable for amplification of the target nucleic acid sequences and hybridization of the amplified target nucleic acid sequences to the capture complex. In some embodiments, the different amplified target nucleic acid sequences hybridize to distinguishable capture complexes. The capture complexes may be, for example, spatially distinguishable or optically distinguishable. In a multiplexed assay, there may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 120, 140, 160, 180, 200, 220, 240, 260, 280, 300, 400, 500, 600, 700, 800, 900, 1000, or any range derivable therein, distinguishable capture complexes combined in a reaction.

In one embodiment, a method for multiplexed amplification of target nucleic acid sequences comprising: providing a plurality of primer pairs, each primer pair comprising: a first primer comprising a target specific sequence, a tag sequence 5′ of the target specific sequence, and a blocker between the target specific sequence and the tag sequence, and a second primer comprising a target specific sequence; providing a reporter attached to either the second primer or to a dNTP; providing a population of capture complexes comprising separate subpopulations of capture complexes, each capture complex in a subpopulation comprising an anti-tag sequence attached to a solid support that distinguish the capture complexes of one subpopulation from the capture complexes of another subpopulation; and combining the plurality of primer pairs, the reporter, the population of capture complexes, and a sample comprising a target nucleic acid sequence under conditions suitable for the amplification of the target nucleic acid sequences and hybridization of the amplified target nucleic acid sequences to the capture complexes. In another embodiment, the invention provides a method for multiplexed amplification of target nucleic acid sequences comprising: providing a plurality of primer pairs, each primer pair comprising: a first primer comprising a target specific sequence, a tag sequence 5′ of the target specific sequence, and a blocker between the target specific sequence and the tag sequence, and a second primer comprising a target specific sequence and a reporter; providing a population of capture complexes comprising separate subpopulations of capture complexes, each capture complex in a subpopulation comprising an anti-tag sequence attached to a solid support that distinguishes the capture complexes of one subpopulation from the capture complexes of another subpopulation; and combining the plurality of primer pairs, the population of capture complexes, and a sample comprising a target nucleic acid sequence under conditions suitable for the amplification of the target nucleic acid sequences and hybridization of the amplified target nucleic acid sequences to the capture complexes. In certain aspects, each first pair in the plurality of primer pairs hybridizes to a different subpopulation of capture complexes.

In one embodiment, the present invention provides a method for amplifying a target nucleic acid sequence comprising: providing a first primer pair comprising: a first primer comprising a target specific sequence, wherein the first primer is covalently coupled to a solid support, a second primer comprising a target specific sequence; a reporter attached to either the second primer or to a dNTP; combining the first primer pair and the reporter with a sample comprising a target nucleic acid sequence under conditions suitable for amplification of the target nucleic acid sequence, wherein the target nucleic acid sequence is amplified. In another embodiment, the present invention provides a method for amplifying a target nucleic acid sequence comprising: providing a first primer pair comprising: a first primer comprising a target specific sequence, wherein the first primer is covalently coupled to a solid support, a second primer comprising a target specific sequence and a reporter; combining the first primer pair with a sample comprising a target nucleic acid sequence under conditions suitable for amplification of the target nucleic acid sequence, wherein the target nucleic acid sequence is amplified. In certain aspects of the invention, a spacer is placed between the first primer and the solid support. The second primer is preferably not covalently coupled to a solid support. The second primer may, however, be associate with the solid support covalently coupled to the first primer by hybridization of the extension product of the second primer with the extension product of the first primer. The same solid supports, reporters, target sequences, and detection methods discussed above, may also be used with this embodiment.

In one embodiment, the present invention provides a method for multiplexed amplification of target nucleic acid sequences comprising: providing a plurality of primer pairs, each primer pair comprising: a first primer comprising a target specific sequence, wherein the first primer is covalently coupled to a solid support of one subpopulation of solid supports that distinguishes the first primer of one primer pair from a first primer of another primer pair, and a second primer comprising a target specific sequence; providing a reporter that is attached to either the second primer or to a dNTP; combining the plurality of primer pairs, the reporter, and a sample comprising a target nucleic acid sequence under conditions suitable for the amplification of the target nucleic acid sequences, wherein the target nucleic acid sequences are amplified. In another embodiment, the present invention provides a method for multiplexed amplification of target nucleic acid sequences comprising: providing a plurality of primer pairs, each primer pair comprising: a first primer comprising a target specific sequence, wherein the first primer is covalently coupled to a solid support of one subpopulation of solid supports that distinguishes the first primer of one primer pair from a first primer of another primer pair, and a second primer comprising a target specific sequence and a reporter; combining the plurality of primer pairs and a sample comprising a target nucleic acid sequence under conditions suitable for the amplification of the target nucleic acid sequences, wherein the target nucleic acid sequences are amplified. In certain aspects of the invention, a spacer is placed between the first primer and the solid support. The second primer is preferably not covalently coupled to a solid support. The second primer may, however, be associate with the solid support covalently coupled to the first primer by hybridization of the extension product of the second primer with the extension product of the first primer. The same solid supports, reporters, target sequences, and detection methods discussed above, may also be used with this embodiment. In certain embodiments, each primer pair in the plurality of primer pairs primes the amplification of a different target specific sequence. In some embodiments, the first primer of each primer pair in the plurality of primer pairs is covalently bound to a different subpopulation of solid supports. In certain aspects, each subpopulation of solid supports is encoded with a fluorescent signal that distinguishes it from other subpopulations of solid supports.

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Schematic Representation of a One-Step Target Detection Assay. The one-step target detection assay uses a tagged and blocked primer (the blocker molecule is represented by the • in the figure) and a direct-labeled primer to amplify a target nucleic acid in the presence of a capture complex comprising beads coupled to anti-tag sequences. The amplification product is directly analyzed on, for example, a flow cytometer or a bead array imaging platform.

FIG. 2. Schematic Representation of a Two-Step Target Detection Assay. The two-step target detection assay uses a tagged and blocked target-specific primer (the blocker molecule is represented by the • in the figure) and a 5′-biotin-labeled target-specific primer in the step 1 PCR. In step 2, the tagged and biotinylated amplification product is concurrently hybridized to the anti-tag sequence coupled to the bead and detected using a SA-PE reporter conjugate.

FIG. 3. Schematic Representation of a Two-Step Genotyping Assay. The two-step genotyping assay uses two tagged and blocked allele-specific primers (the blocker molecule is represented by the • in the figure) and a 5′-biotin-labeled target-specific primer in the step 1 PCR. As shown in step 1, only the allele-specific primer with C at its 3′ end is extended because it is complementary to the G in the target genomic DNA. Each allele-specific primer has a unique tag (Tag 1 or Tag 2). In step 2, the tagged and biotinylated amplification product is concurrently hybridized to the anti-tag sequence coupled to the bead and detected using a SA-PE reporter conjugate.

FIG. 4. Schematic Representation of a Two-Step Genotyping Assay Using an Allele-Specific Primer with an Internal Discriminating Nucleotide. Amplification of a nucleic acid target is performed with two tagged and blocked allele-specific PCR primers (the blocker molecule is represented by the • in the figure) and a 5′-biotin-labeled target-specific PCR primer. Additionally, the discriminating nucleotide for the two allele-specific primers is internal (i.e., it is not at the 3′ end of the primer). As shown in step 1, only the allele-specific primer with C as its discriminating nucleotide is extended because it is complementary to the G in the target genomic DNA. Each allele-specific primer has a unique tag (Tag 1 or Tag 2). In step 2, the tagged and biotinylated amplification product is concurrently hybridized to the anti-tag sequence coupled to the bead and detected using a SA-PE reporter conjugate.

FIG. 5. Schematic Representation of a Tag Sequence Orientation not Extendable by the Polymerase Enzyme. The tagged primer is shown with the tag sequence in the 3′ to 5′ orientation and the target specific sequence in the 5′ to 3′ orientation. The orientation of the tag prevents extension of the polymerase enzyme into the tag sequence in step 1. Accordingly, the tag sequence remains single-stranded and available for hybridization to the anti-tag sequence in step 2.

FIG. 6. Schematic Representation of a One-Step Target Detection Assay in Which a Target-Specific Primer is Directly Coupled to the Bead. In this embodiment of the one-step target detection assay PCR is performed using a target-specific primer directly coupled to the bead and a direct-labeled primer to amplify a target nucleic acid. The amplification product is directly analyzed on, for example, a flow cytometer or a bead array imaging platform.

FIGS. 7A to 7F. Two-Step, Multiplexed Detection of Prognostic Indicators of Recovery from Inflammatory Conditions. FIGS. 7A and 7B show the results of the 3-plex assay on sample NA06985. FIGS. 7C and 7D show the results of the 3-plex assay on sample NA07022. FIGS. 7E and 7F show the results of the 3-plex assay on sample NA07055.

FIG. 8. Detection of Coxsackie Virus. FIG. 8 shows a comparison of the MFI (Median Fluorescence Intensity) for Coxsackie virus detection in a one-step assay format and a two-step assay format.

FIGS. 9A to 9D. One-Step, Multiplexed Detection of Viruses. Multiplexed RT-PCR was carried out to detect Adeno, Flu B, Enterovirus/Rhinovirus, RSV A and the internal control, MS2. Each of the primer pairs included two target-specific PCR primers. One primer contained a unique universal sequence (tag) on the 5′ end and a blocker molecule (iSp18) positioned between the tag and target specific regions of the primer. The other primer was 5′ labeled with a fluorophore (either HEX, Cy3 or Alexa532) or biotin. The biotin version of the assay, which served as the control format, was carried out as a 2-step assay. FIG. 9A shows the results for coxsackie virus. FIG. 9B shows the results for Adenovirus. FIG. 9C shows the results for RSV A. FIG. 9D shows the results for Flu B.

### DESCRIPTION OF ILLUSTRATIVE EMBODIMENTS

### A. Nucleic Acid Target Detection

The methods and compositions of the present invention significantly simplify genetic analysis by methods such as allele-specific primer extension (ASPE) and allele-specific PCR (AS-PCR). For example, in one embodiment the present invention provides a one-step method for amplifying and detecting a target nucleic acid sequence by (1) providing a first primer pair, wherein one primer of the primer pair comprises a target specific sequence, a tag sequence 5′ of the target specific sequence, and a blocker between the target specific sequence and the tag sequence, and the other primer of the primer pair comprises a target specific sequence and a reporter (e.g., fluorophore); (2) providing a capture complex comprising an anti-tag sequence attached to a solid support; (3) combining the first primer pair, the capture complex, and a sample comprising a target nucleic acid sequence under conditions suitable for amplification of the target nucleic acid sequence and hybridization of the amplified target nucleic acid sequence to the capture complex; and (4) detecting the amplified target nucleic acid sequence. This embodiment may be employed in, for example, AS-PCR. In addition, this embodiment may be used in a multiplex format.

The one-step amplification and detection method of the present invention may be used, for example, to reduce the number of assay steps in the current commercially available Luminex Tag-It® technology platform from five steps to a single-step. This results in an assay that is (a) much simpler to perform, (b) less prone to amplicon contamination, (c) does not require Exonuclease 1 (Exo) and shrimp alkaline phosphatase (SAP) treatment of the PCR product (a step that is prone to user error) and (d) more adaptable to automation. Furthermore, the assay turn-around-time for the Luminex Tag-It® platform may be reduced from approximately 5-6 hours to 2-3 hours (depending on whether reverse-transcription is required). Accordingly, the one-step method allows for assay turn-around-times that are comparable to those of real-time PCR assays but in addition are capable of multiplexing at levels (greater than 4-6-plex) that are not currently achievable by real-time PCR technology, due to the requirement for multiple, distinguishable fluors in real-time PCR technology.

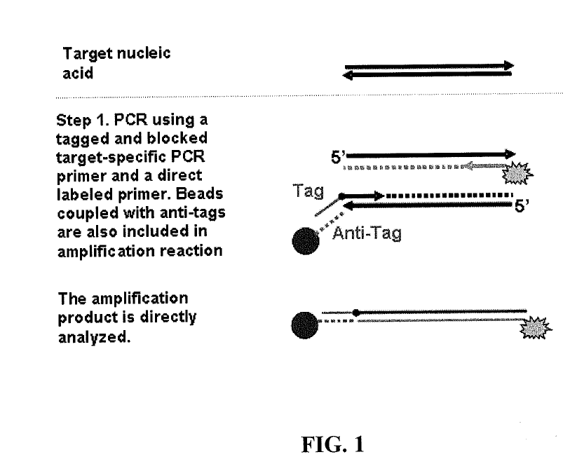

FIG. 1 provides a schematic representation of a one-step target detection assay in which an encoded bead is the solid support in the capture complex. Amplification and detection of a nucleic acid target is performed with a tagged and blocked target-specific PCR primer and a 5′-direct-labeled target-specific PCR primer. Labeled beads coupled to anti-tag sequences are also included in the amplification reaction. Following the amplification reaction, detection of the labeled, partially double-stranded product is carried out on, for example, a Luminex instrument such as the Luminex 100™ IS system or Luminex 200™ System. The target nucleic acid can be single-stranded or double-stranded. If the target nucleic acid is RNA, then RT-PCR is performed. Simultaneous analysis of multiple different targets (multiplex) may be performed using multiple primer sets where one primer from each set is tagged with a unique universal sequence (tag). FIG. 1 illustrates the method being used in a format that indicates the presence or absence of a target sequence, but a person of ordinary skill in the art would understand that the method may also be employed in a genotyping assay. For example, for an AS-PCR genotyping assay the primers would be designed to be allele-specific. An allele-specific primer has a discriminating nucleotide. The discriminating nucleotide may be at the 3′ terminus of the primer or it may be internal (i.e., not at the 3′ terminus). In certain aspects of the invention the internal, allele-discriminating nucleotide is at the second, third, fourth, or fifth nucleotide from the 3′ terminus of the primer.

It is surprising that in both the one-step and two step multiplex assay formats described herein, results of sufficient specificity to detect one or more target nucleic acids present in a sample, are obtained. Since unextended tagged primers are not removed prior to hybridization and detection, one would expect that unextended primers would out-compete labeled extension products for binding to encoded beads through their corresponding anti-tag sequences, resulting in insufficient specific signal for target detection. It is also surprising that in embodiments using biotinylated primers results were obtained with sufficient specificity to detect one or more target nucleic acids present in a sample. This is surprising because one would have expected that the competition between the biotinylated primers and the biotinylated amplification products for the limited streptavidin-phycoerythrin would have resulted in an insufficient specific signal for target detection.

In another embodiment, the present invention provides a two-step method for amplifying and detecting a target nucleic acid sequence by (1) providing a first primer pair, wherein one primer of the primer pair comprises a target specific sequence, a tag sequence 5′ of the target specific sequence, and a blocker between the target specific sequence and the tag sequence, and the other primer of the primer pair comprises a target specific sequence and a reporter (e.g., biotin); (2) combining the first primer pair and a sample comprising a target nucleic acid sequence under conditions suitable for amplification of the target nucleic acid sequence; (3) hybridization the amplified target nucleic acid sequence to a capture complex comprising an anti-tag sequence attached to a solid support; and (4) detecting the amplified target nucleic acid sequence. The hybridization and detection steps may be performed concurrently. This embodiment may be used in a multiplex format.

The two-step amplification and detection method of the present invention may be used, for example, to reduce the number of assay steps in the current commercially available Luminex Tag-It® technology platform from five steps to two steps. This reduction of steps results in an assay that is (a) much simpler to perform, (b) less prone to amplicon contamination (requiring a maximum of one liquid transfer step), (c) does not require Exonuclease 1 (Exo) and shrimp alkaline phosphatase (SAP) treatment of the PCR product (a step that is prone to user error) and (d) more adaptable to automation. Furthermore, the two-step method allows for assay turn-around-times that are comparable to those of real-time PCR assays but in addition are capable of multiplexing at levels (greater than 4-6-plex) that are not currently achievable by real-time PCR technology.

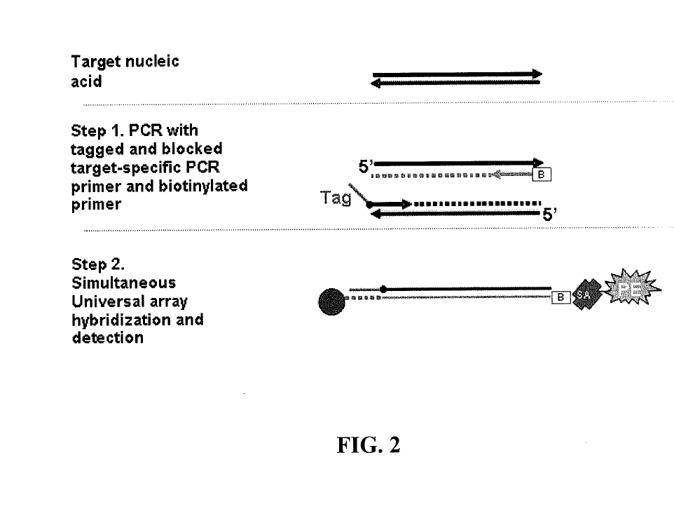

FIG. 2 provides a schematic representation of a two-step target detection assay in which a labeled bead is the solid support in the capture complex. As shown in FIG. 2, amplification of a nucleic acid target is performed with a tagged and blocked target-specific PCR primer and a 5′-biotin-labeled target-specific PCR primer. The resultant partially double-stranded, tagged and labeled (biotinylated) amplification product is then simultaneously sorted through hybridization to anti-tag sequences coupled onto labeled beads and detected using a streptavidin-phycoerythrin (SA-PE) reporter conjugate. The target nucleic acid can be single-stranded or double-stranded. If the target nucleic acid is RNA, then RT-PCR is performed. Simultaneous analysis of multiple different targets (multiplex) may be performed using multiple primer sets where one primer from each set is tagged with a unique universal sequence (tag). FIG. 2 illustrates the method being used in an assay format that indicates the presence or absence of a target sequence, but a person of ordinary skill in the art would understand that the method may also be employed in a genotyping assay. For example, for an AS-PCR genotyping assay the primers would be designed to be allele-specific.

In a further embodiment, the present invention provides a two-step method for genotyping a target nucleic acid sequence by (1) providing a first primer pair, wherein one primer of the primer pair comprises an allele-specific sequence, a tag sequence 5′ of the allele-specific sequence, and a blocker between the target specific sequence and the tag sequence, and the other primer of the primer pair comprises a target specific sequence and a reporter (e.g., biotin); (2) combining the first primer pair and a sample comprising a target nucleic acid sequence under conditions suitable for amplification of the target nucleic acid sequence; (3) hybridization the amplified target nucleic acid sequence to a capture complex comprising an anti-tag sequence attached to a solid support; and (4) detecting the amplified target nucleic acid sequence. The hybridization and detection steps may be performed concurrently. It should be noted that while in this example it is the tagged and blocked primer that is the allele-specific primer, in an alternative embodiment the reporter (e.g., biotin) labeled primer could be the allele-specific primer, provided each allele specific primer was distinguishable on the basis of a different reporter. Furthermore, the primer pair could be a primer set containing, for example, allele-specific primers for the wild-type allele and one or more mutant/polymorphic alleles. This embodiment may be used in a multiplex format.

The two-step genotyping method of the present invention may be used, for example, to reduce the number of assay steps in the current commercially available Luminex Tag-It® technology platform from five steps to two steps. This reduction of steps results in an assay that is (a) much simpler to perform, (b) less prone to amplicon contamination (requiring a maximum of one liquid transfer step), (c) does not require Exonuclease 1 (Exo) and shrimp alkaline phosphatase (SAP) treatment of the PCR product (a step that is prone to user error) and (d) more adaptable to automation. Furthermore, the two-step method allows for assay turn-around-times that are comparable to those of real-time PCR assays but in addition are capable of multiplexing at levels (greater than 4-6-plex) that are not currently achievable by real-time PCR technology.

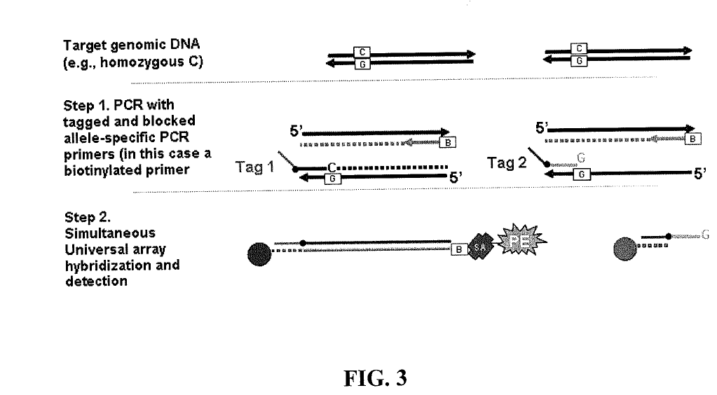

FIG. 3 provides a schematic representation of a two-step genotyping assay in which a labeled bead is the solid support in the capture complex. As shown in FIG. 3, amplification of a nucleic acid target is performed with two tagged and blocked allele-specific PCR primers (one for the C allele and one for the G allele) and a 5′-biotin-labeled target-specific PCR primer. Each allele-specific primer has a unique tag (Tag 1 and Tag 2 in FIG. 3). Since the target genomic DNA is homozygous C, only one of the two allele-specific primers is able to be extended. The resultant partially double-stranded, tagged and labeled (biotinylated) amplification product is then simultaneously sorted through hybridization to anti-tag sequences coupled onto labeled beads and detected using a streptavidin-phycoerythrin (SA-PE) reporter conjugate. Simultaneous genotyping at multiple mutation/polymorphic sites (multiplex) may be performed using multiple primer sets where one primer from each set is tagged with a unique universal sequence (tag). It is not necessary to wash away the free (i.e., unincorporated into amplification products) primers prior to the hybridization and detection step.

In another embodiment, the present invention provides a two-step method for genotyping a target nucleic acid sequence that uses an allele-specific primer with an internal discriminating nucleotide. This is in contrast to conventional allele-discrimination based on a primer's most 3′ nucleotide. This method comprises (1) providing a first primer pair, wherein one primer of the primer pair comprises an allele-specific sequence wherein the allele-discriminating nucleotide is not at the 3′ terminus of the primer, a tag sequence 5′ of the allele-specific sequence, and a blocker between the target specific sequence and the tag sequence, and the other primer of the primer pair comprises a target specific sequence and a reporter (e.g., biotin); (2) combining the first primer pair and a sample comprising a target nucleic acid sequence under conditions suitable for amplification of the target nucleic acid sequence; (3) hybridization the amplified target nucleic acid sequence to a capture complex comprising an anti-tag sequence attached to a solid support; and (4) detecting the amplified target nucleic acid sequence. The hybridization and detection steps may be performed concurrently. It should be noted that while in this example it is the tagged and blocked primer that is the allele-specific primer, in an alternative embodiment the reporter (e.g., biotin) labeled primer could be the allele-specific primer, provided each allele specific primer was distinguishable on the basis of a different reporter. Furthermore, the primer pair could be a primer set containing, for example, allele-specific primers for the wild-type allele and one or more mutant/polymorphic alleles. As mentioned above, the allele-specific primer has an internal (i.e., not at the 3′ terminus) discriminating nucleotide. In certain aspects of the invention the internal, allele-discriminating nucleotide is at the second, third, fourth, or fifth nucleotide from the 3′ terminus of the primer. This embodiment may be used in a multiplex format.

This two-step genotyping method using an allele-specific primer with an internal discriminating nucleotide may be used, for example, to reduce the number of assay steps in the current commercially available Luminex Tag-It® technology platform from five steps to two steps. This reduction of steps results in an assay that is (a) much simpler to perform, (b) less prone to amplicon contamination (requiring a maximum of one liquid transfer step), (c) does not require Exonuclease 1 (Exo) and shrimp alkaline phosphatase (SAP) treatment of the PCR product (a step that is prone to user error) and (d) more adaptable to automation. Furthermore, the two-step method allows for assay turn-around-times that are comparable to those of real-time PCR assays but in addition are capable of multiplexing at levels (greater than 4-6-plex) that are not currently achievable by real-time PCR technology.

FIG. 4 provides a schematic representation of a two-step genotyping assay using an allele-specific primer with an internal discriminating nucleotide in which a labeled bead is the solid support in the capture complex. As shown in FIG. 4, amplification of a nucleic acid target is performed with two tagged and blocked allele-specific PCR primers (one for the C allele and one for the G allele) and a 5′-biotin-labeled target-specific PCR primer. Each allele-specific primer has a unique tag (Tag 1 and Tag 2 in FIG. 4). Additionally, the discriminating nucleotide for the two allele-specific primers is internal (i.e., it is not at the 3′ end of the primer). Since the target genomic DNA is homozygous C, only one of the two allele-specific primers is able to be extended. The resultant partially double-stranded, tagged and labeled (biotinylated) amplification product is then simultaneously sorted through hybridization to anti-tag sequences coupled onto labeled beads and detected using a streptavidin-phycoerythrin (SA-PE) reporter conjugate. Simultaneous genotyping at multiple mutation/polymorphic sites (multiplex) may be performed using multiple primer sets where one primer from each set is tagged with a unique universal sequence (tag). It is not necessary to wash away the free (i.e., unincorporated into amplification products) primers prior to the hybridization and detection step. It should be noted that while in this example it is the tagged and blocked primer that is the allele-specific primer, in an alternative embodiment the reporter (e.g., biotin) labeled primer could be the allele-specific primer, provided each allele specific primer was distinguishable on the basis of a different reporter.

FIG. 6 provides a schematic representation of a one-step target detection assay in which a target-specific primer is directly coupled to the bead. In this embodiment of the one-step target detection assay PCR is performed using a target-specific primer directly coupled to the bead and a direct-labeled primer to amplify a target nucleic acid. Since one of the primers is directly coupled to the bead, a primer that is tagged and blocked is not required. Likewise, anti-tag sequences coupled to the beads also are not required since the primer is directly coupled to the bead. The primer may be directly coupled to the bead with, for example, an amino group on the primer that binds a carboxyl group on the bead. A spacer may be placed between the primer and the bead to reduce steric hindrance. Following the amplification reaction, detection of the labeled, double-stranded product is carried out on, for example, a Luminex instrument such as the Luminex 100™ IS system or Luminex 200™ System. The target nucleic acid can be single-stranded or double-stranded. If the target nucleic acid is RNA, then RT-PCR is performed. Simultaneous analysis of multiple different targets (multiplex) may be performed by using multiple primer sets and multiple distinguishable subpopulations of beads, wherein each primer set is associated with a uniquely identifiable subpopulation of beads. FIG. 6 illustrates the method being used in a format that indicates the presence or absence of a target sequence, but a person of ordinary skill in the art would understand that the method may also be employed in a genotyping assay. For example, for an AS-PCR genotyping assay the primers would be designed to be allele-specific. An allele-specific primer has a discriminating nucleotide. The discriminating nucleotide may be at the 3′ terminus of the primer or it may be internal (i.e., not at the 3′ terminus). In certain aspects of the invention the internal, allele-discriminating nucleotide is at the second, third, fourth, or fifth nucleotide from the 3′ terminus of the primer.

While the preceding examples have been discussed generally in the context of their use with the Luminex Tag-It® technology platform, it will be understood by those in the art that the methods and compositions of the present invention may also be used in connection with other methods or platforms for ASPE, AS-PCR, and the detection of target sequences. ASPE technology has been generally described in U.S. Pat. No. 4,851,331, which is incorporated herein by reference. Variations of ASPE and AS-PCR have been described, for example, in U.S. Pat. No. 6,287,778, PCT Application (WO 00/47766), U.S. Pat. No. 5,595,890, U.S. Pat. No. 5,639,611, and U.S. Pat. No. 5,137,806, each of which in incorporate herein by reference.

In addition, while the preceding examples use a direct-labeled primer, the methods can be performed with labeled dNTPs in the amplification reaction. If labeled dNTPs are used, then the primer(s) do not need to be labeled.

It is surprising that in both the one-step and two step multiplex assay formats described herein, results of sufficient sensitivity and specificity to detect one or more target nucleic acids present in a sample, are obtained. Since unextended tagged primers are not removed prior to hybridization and detection, one would expect that unextended primers would out-compete labeled extension products for binding to encoded beads through their corresponding anti-tag sequences, resulting in insufficient sensitivity for target detection. For this reason, removal of unextended primers is usually recommended. In the present invention, this step is not required. Furthermore, in the one-step format, it is well known that primer extension occurs with decreased efficiency on solid substrates. It is thus even more surprising that sufficient sensitivity was achieved in this format. It is also surprising that in embodiments using biotinylated primers results were obtained with sufficient specificity to detect one or more target nucleic acids present in a sample. This is surprising because one would have expected that the competition between the biotinylated primers and the biotinylated amplification products for the limited streptavidin-phycoerythrin would have resulted in an insufficient specific signal for target detection.

### B. Nucleic Acids

1. Preparation of Nucleic Acids

The tag sequences, anti-tag sequences, and primers disclosed herein may be prepared by any technique known to one of ordinary skill in the art, such as for example, chemical synthesis, enzymatic production, or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al., 2001, incorporated herein by reference).

Nucleic acids used as a template for amplification may be isolated from cells, tissues or other samples according to standard methodologies (Sambrook et al., 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

A reverse transcriptase PCR™ amplification procedure may be performed to reverse transcribe mRNA into cDNA. Methods of RT-PCR are well known in the art (see Sambrook et al., 2001). Alternative methods for RT-PCR utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of nucleic acid sequences that may be used in the practice of certain aspects of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence, which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids, which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA).

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR™” (Frohman, 1990; Ohara et al., 1989).

Amplification products may be visualized. If the amplification products are integrally labeled with radio- or fluorescent-labeled nucleotides, the separated amplification products can be exposed to x-ray film or visualized under the appropriate excitatory spectra. In another approach, a labeled nucleic acid probe is brought into contact with the amplified marker sequence, following separation of the amplification products. The probe may be conjugated to, for example, a chromophore, fluorophore, radiolabel, or conjugated to a binding partner, such as an antibody or biotin.

Various nucleic acid detection methods known in the art are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

2. Nucleic Acid Analogs

A nucleic acid sequence may comprise, or be composed entirely of, an analog of a naturally occurring nucleotide. As used herein an “analog” refers to a molecule that may or may not structurally resemble a naturally occurring molecule or moiety, but possesses similar functions.

Nucleotide analogs are well known in the art. A non-limiting example is a “peptide nucleic acid,” also known as a “PNA,” “peptide-based nucleic acid analog,” or “PENAM,” described in U.S. Pat. Nos. 5,786,461, 5,891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (Egholm et al., 1993; PCT/EP/01219). A peptide nucleic acid generally comprises one or more nucleotides or nucleosides that comprise a nucleobase moiety, a nucleobase linker moiety that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

Another non-limiting example is a locked nucleic acid or “LNA.” An LNA monomer is a bi-cyclic compound that is structurally similar to RNA nucleosides. LNAs have a furanose conformation that is restricted by a methylene linker that connects the 2′-O position to the 4′-C position, as described in Koshkin et al., 1998a and 1998b and Wahlestedt et al., 2000.

Yet another non-limiting example is a “polyether nucleic acid,” described in U.S. Pat. No. 5,908,845, incorporated herein by reference. In a polyether nucleic acid, one or more nucleobases are linked to chiral carbon atoms in a polyether backbone.

3. Hybridization

Sequence-specific nucleic acid hybridization assays are used for the detection of specific genetic sequences as indicators of genetic anomalies, mutations, and disease propensity. In addition, they are used for the detection of various biological agents and infectious pathogens. As used herein, “hybridization,” “hybridizes” or “capable of hybridizing” is understood to mean the forming of a double or triple stranded molecule or a molecule with partial double or triple stranded nature. The term “anneal” as used herein is synonymous with “hybridize.” The term “hybridization,” “hybridizes” or “capable of hybridizing” encompasses the terms “stringent conditions” or “high stringency” and the terms “low stringency” or “low stringency conditions.”

As used herein “stringent conditions” or “high stringency” are those conditions that allow hybridization between or within one or more nucleic acid strands containing complementary sequences, but preclude hybridization of non-complementary sequences. Such conditions are well known to those of ordinary skill in the art, and are preferred for applications requiring high selectivity. Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acids, the length and nucleobase content of the target sequences, the charge composition of the nucleic acids, and to the presence or concentration of formamide, tetramethylammonium chloride or other solvents in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Non-limiting examples of low stringency conditions include hybridization performed at about 0.15 M to about 0.9 M NaCl at a temperature range of about 20° C. to about 50° C. Of course, it is within the skill of one in the art to further modify the low or high stringency conditions to suite a particular application.

### C. Detection of Nucleic Acids

1. Labels

To detect nucleic acids, it will be advantageous to employ nucleic acids in combination with an appropriate detection means. Recognition moieties incorporated into primers, incorporated into the amplified product during amplification, or attached to probes are useful in the identification of nucleic acid molecules. A number of different labels may be used for this purpose such as fluorophores, chromophores, radiophores, enzymatic tags, antibodies, chemi/electroluminescent labels, affinity labels, etc. One of skill in the art will recognize that these and other labels not mentioned herein can be used with success in this invention. Examples of affinity labels include, but are not limited to the following: an antibody, an antibody fragment, a receptor protein, a hormone, biotin, digoxigen, DNP, or any polypeptide/protein molecule that binds to an affinity label.

Examples of enzyme tags include enzymes such as urease, alkaline phosphatase or peroxidase to mention a few. Colorimetric indicator substrates can be employed to provide a detection means visible to the human eye or spectrophotometrically, to identify specific hybridization with complementary nucleic acid-containing samples. All of these examples are generally known in the art and the skilled artisan will recognize that the invention is not limited to the examples described above.

Examples of fluorophores include, a red fluorescent squarine dye such as 2,4-Bis[1,3,3-trimethyl-2-indolinylidenemethyl]cyclobutenediylium-1,3-dioxolate, an infrared dye such as 2,4 Bis[3,3-dimethyl-2-(1H-benz[e]indolinylidenemethyl)]cyclobutenediylium-1,3-dioxolate, or an orange fluorescent squarine dye such as 2,4-Bis[3,5-dimethyl-2-pyrrolyl]cyclobutenediylium-1,3-diololate. Additional non-limiting examples of fluorophores include quantum dots, Alexa Fluor® dyes, AMCA, BODIPY® 630/650, BODIPY® 650/665, BODIPY®-FL, BODIPY®—R6G, BODIPY®-TMR, BODIPY®-TRX, Cascade Blue®, CyDye™, including but not limited to Cy2™, Cy3™, and Cy5™, a DNA intercalating dye, 6-FAM™, Fluorescein, HEX™, 6-JOE, Oregon Green® 488, Oregon Green® 500, Oregon Green® 514, Pacific Blue™, REG, phycobilliproteins including, but not limited to, phycoerythrin and allophycocyanin, Rhodamine Green™, Rhodamine Red™, ROX™, TAMRA™, TET™, Tetramethylrhodamine, or Texas Red®. A signal amplification reagent, such as tyramide (PerkinElmer), may be used to enhance the fluorescence signal.

2. Gene Chips and Microarrays

The capture complexes of the present invention may comprise a gene chip or microarray. Arrays and gene chip technology provide a means of rapidly screening a large number of nucleic acid samples for their ability to hybridize to a variety of single stranded oligonucleotide probes immobilized on a solid substrate. These techniques involve quantitative methods for analyzing large numbers of genes rapidly and accurately. The technology capitalizes on the complementary binding properties of single stranded DNA to screen DNA samples by hybridization (Pease et al., 1994; Fodor et al., 1991). Basically, an array or gene chip consists of a solid substrate upon which an array of single stranded DNA or RNA molecules have been attached. For screening, the chip or array is contacted with a single stranded DNA or RNA sample, which is allowed to hybridize under stringent conditions. The chip or array is then scanned to determine which probes have hybridized.

The ability to directly synthesize on or attach polynucleotide probes to solid substrates is well known in the art. See U.S. Pat. Nos. 5,837,832 and 5,837,860, both of which are expressly incorporated by reference. A variety of methods have been utilized to either permanently or removably attach the probes to the substrate. Exemplary methods include: the immobilization of biotinylated nucleic acid molecules to avidin/streptavidin coated supports (Holmstrom, 1993), the direct covalent attachment of short, 5′-phosphorylated primers to chemically modified polystyrene plates (Rasmussen et al., 1991), or the precoating of the polystyrene or glass solid phases with poly-L-Lys or poly L-Lys, Phe, followed by the covalent attachment of either amino- or sulfhydryl-modified oligonucleotides using bi-functional crosslinking reagents (Running et al., 1990; Newton et al., 1993). When immobilized onto a substrate, the probes are stabilized and therefore may be used repeatedly. In general terms, hybridization is performed on an immobilized nucleic acid target or a probe molecule that is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules.

3. Luminex xMAP Technology

In certain embodiments, the present invention is used in conjunction with Luminex® xMAP® technology. The Luminex technology allows the detection of nucleic acid products immobilized on fluorescently encoded microspheres. By dyeing microspheres with 10 different intensities of each of two spectrally distinct fluorochromes, 100 fluorescently distinct populations of microspheres are produced. These individual populations (sets) can represent individual detection sequences and the magnitude of hybridization on each set can be detected individually. The magnitude of the hybridization reaction is measured using a third reporter, which is typically a third spectrally distinct fluorophore. The reporter molecule signals the extent of the reaction by attaching to the molecules on the microspheres. As both the microspheres and the reporter molecules are labeled, digital signal processing allows the translation of signals into real-time, quantitative data for each reaction. The Luminex technology is described, for example, in U.S. Pat. Nos. 5,736,330, 5,981,180, and 6,057,107, all of which are specifically incorporated by reference.

4. Competitive Binding Assays

The present invention may also be used in conjunction with a competitive binding assay format. In general, this format involves a sequence coupled to a solid surface, and a labeled sequence, which is complementary to the sequence coupled to the solid surface, in solution. With this format, the target sequence in the sample being assayed does not need to be labeled. Rather, the target sequence's presence in the sample is detected because it competes with the labeled complement for hybridization with the immobilized detection sequence. Thus, if the target sequence is present in the sample, the signal decreases as compared to a sample lacking the target sequence.

The Luminex xMAP technology described above can be used in a competitive binding assay format. The use of the Luminex technology in a competitive binding assay format is described in U.S. Pat. Nos. 5,736,330 and 6,057,107, incorporated herein by reference.

5. Flow Cytometry

Flow cytometry is a useful tool in the analysis of biomolecules. In the context of the present invention, flow cytometry is particularly useful in the analysis of microsphere based assays, such as the Luminex xMAP® system. Flow cytometry involves the separation of cells or other particles, such as microspheres, in a liquid sample. Generally, the purpose of flow cytometry is to analyze the separated particles for one or more characteristics. The basic steps of flow cytometry involve the direction of a fluid sample through an apparatus such that a liquid stream passes through a sensing region. The particles should pass one at a time by the sensor and are categorized based on size, refraction, light scattering, opacity, roughness, shape, fluorescence, etc.

In the context of the Luminex xMAP system, flow cytometry can be used for simultaneous sequence identification and hybridization quantification. Internal dyes in the microspheres are detected by flow cytometry and used to identify the specific nucleic acid sequence to which a microsphere is coupled. The label on the target nucleic acid molecule is also detected by flow cytometry and used to quantify target hybridization to the microsphere.

Methods of flow cytometry are well know in the art and are described, for example, in U.S. patents, all of which are specifically incorporated by reference. U.S. Pat. Nos. 5,981,180, 4,284,412; 4,989,977; 4,498,766; 5,478,722; 4,857,451; 4,774,189; 4,767,206; 4,714,682; 5,160,974; and 4,661,913

6. Bead Arrays

Microsphere based assays may also be analyzed on bead array platforms. In general, bead array platforms image beads and analytes distributed on a substantially planar array. In this way, imaging of bead arrays is similar to the gene chips discussed above. However, in contrast to gene chips where the analyte is identified by its spatial position on the array, bead arrays typically identify the analyte by the encoded microsphere to which it is bound. Examples of commercially available bead array systems include Illumina's BeadXpress™ Reader and BeadStation 500™.

7. Tag Sequences

As mentioned above, various aspects of the present invention use complementary tag sequences (i.e., tags and anti-tags) in the primers and capture molecules. A number of approaches have been developed that involve the use of oligonucleotide tags attached to a solid support that can be used to specifically hybridize to the tag complements that are coupled to primers, probe sequences, target sequences, etc. The proper selection of non-hybridizing tag and anti-tag sequences is useful in assays, particularly assays in a highly parallel hybridization environment, that require stringent non-cross hybridizing behavior.

Certain thermodynamic properties of forming nucleic acid hybrids are considered in the design of tag and anti-tag sequences. The temperature at which oligonucleotides form duplexes with their complementary sequences known as the Tₘ (the temperature at which 50% of the nucleic acid duplex is dissociated) varies according to a number of sequence dependent properties including the hydrogen bonding energies of the canonical pairs A-T and G-C (reflected in GC or base composition), stacking free energy and, to a lesser extent, nearest neighbor interactions. These energies vary widely among oligonucleotides that are typically used in hybridization assays. For example, hybridization of two probe sequences composed of 24 nucleotides, one with a 40% GC content and the other with a 60% GC content, with its complementary target under standard conditions theoretically may have a 10° C. difference in melting temperature (Mueller et al., 1993). Problems in hybridization occur when the hybrids are allowed to form under hybridization conditions that include a single hybridization temperature that is not optimal for correct hybridization of all oligonucleotide sequences of a set. Mismatch hybridization of non-complementary probes can occur forming duplexes with measurable mismatch stability (Santalucia et al., 1999). Mismatching of duplexes in a particular set of oligonucleotides can occur under hybridization conditions where the mismatch results in a decrease in duplex stability that results in a higher Tₘ than the least stable correct duplex of that particular set. For example, if hybridization is carried out under conditions that favor the AT-rich perfect match duplex sequence, the possibility exists for hybridizing a GC-rich duplex sequence that contains a mismatched base having a melting temperature that is still above the correctly formed AT-rich duplex. Therefore, design of families of oligonucleotide sequences that can be used in multiplexed hybridization reactions must include consideration for the thermodynamic properties of oligonucleotides and duplex formation that will reduce or eliminate cross hybridization behavior within the designed oligonucleotide set.

There are a number of different approaches for selecting tag and anti-tag sequences for use in multiplexed hybridization assays. The selection of sequences that can be used as zip codes or tags in an addressable array has been described in the patent literature in an approach taken by Brenner and co-workers (U.S. Pat. No. 5,654,413, incorporated herein by reference). Chetverin et al. (WO 93/17126, U.S. Pat. Nos. 6,103,463 and 6,322,971, incorporated herein by reference) discloses sectioned, binary oligonucleotide arrays to sort and survey nucleic acids. These arrays have a constant nucleotide sequence attached to an adjacent variable nucleotide sequence, both bound to a solid support by a covalent linking moiety. Parameters used in the design of tags based on subunits are discussed in Barany et al. (WO 9731256, incorporated herein by reference). A multiplex sequencing method has been described in U.S. Pat. No. 4,942,124, incorporated herein by reference. This method uses at least two vectors that differ from each other at a tag sequence.

U.S. Pat. No. 7,226,737, incorporated herein by reference, describes a set of 210 non-cross hybridizing tags and anti-tags. U.S. Published Application No. 2005/0191625, incorporated herein by reference, discloses a family of 1168 tag sequences with a demonstrated ability to correctly hybridize to their complementary sequences with minimal cross hybridization.

A population of oligonucleotide tag or anti-tag sequences may be conjugated to a population of primers or other polynucleotide sequences in several different ways including, but not limited to, direct chemical synthesis, chemical coupling, ligation, amplification, and the like. Sequence tags that have been synthesized with primer sequences can be used for enzymatic extension of the primer on the target for example in PCR amplification. A population of oligonucleotide tag or anti-tag sequences may be conjugated to a solid support by, for example, surface chemistries on the surface of the support.

8. Blocker Moieties

As discussed above, one primer of a primer pair used in an amplification reaction comprises a tag sequence. Following the initial extension of the primer comprising the tag sequence, the tagged extension product may serve as a template for the other primer of the primer pair. It would be undesirable, however, for the extension on such a template to proceed through the tag region as this would interfere with the hybridization of the tag sequence with the anti-tag sequence of the capture complex. Accordingly, a blocker is positioned between the target specific sequence and the tag sequence of the primer. Blocker moieties prevent the polymerase from extending into the tag sequence region, which allows the tag sequence to remain single-stranded during amplification and therefore free to hybridize to its complementary anti-tag sequence in the capture complex.

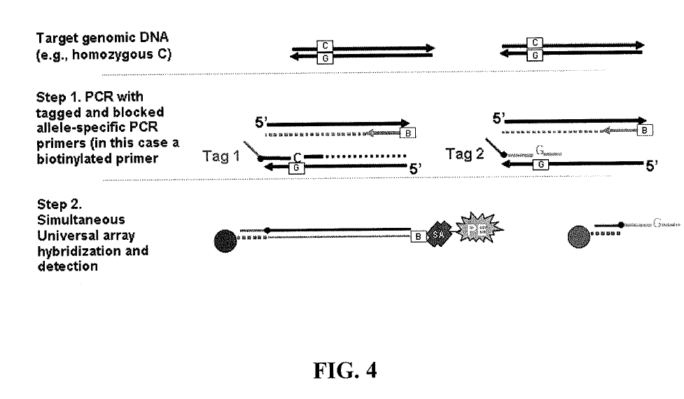

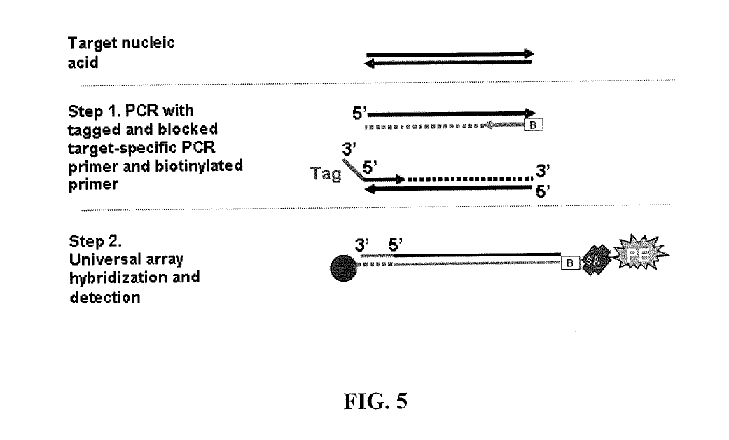

A blocker moiety refers to any moiety that when linked (e.g., covalently linked) between a first nucleotide sequence and a second nucleotide sequence is effective to inhibit and preferably prevent extension of either the first or second nucleotide sequence but not both the first and second nucleotide sequence. There are a number of molecules that may be used as blocker moieties. Non-limiting examples of blocker moieties include C6-20 straight chain alkylenes and iSp18 (which is an 18-atom hexa-ethyleneglycol). Blocker moieties may include, for example, at least one deoxy ribofuranosyl naphthalene or ribofuranosyl naphthalene moiety, which may be linked to the adjacent nucleotides via a 3′-furanosyl linkage or preferably via a 2′-furanosyl linkage. A blocker moiety may be an oligonucleotide sequence that is in the opposite orientation as the target specific sequence. Accordingly, in certain aspects of the invention a primer's tag sequence may be both the tag and the blocker. This is illustrated in FIG. 5 where the target specific sequence is in the 5′ to 3′ orientation but the tag sequence is in the 3′ to 5′ orientation. In this orientation the tag sequence is not extendable by the polymerase enzyme. Various blocker moieties and their use are described in U.S. Pat. No. 5,525,494, which is incorporated herein by reference.

If a blocker moiety is not used, steps may be taken to permit the hybridization of the tag sequence with the anti-tag sequence. For example, an anti-tagged primer may be substituted for the tagged and blocked primer in the amplification. In which case, the polymerase is allowed to extend into the anti-tag sequence region, creating the complementary tag sequence. The double-stranded amplification product which contains an anti-tag/tag region is then denatured prior to hybridization to the anti-tag coupled to the solid substrate.

### D. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### 1. Example 1

### Two-Step Target Detection Assay

A multiplex assay was designed in the 2-step format described herein to identify alleles that have been identified as prognostic indicators of recovery from inflammatory conditions, for example, SIRS (systemic inflammatory response syndrome). Specifically, a 3-plex assay was designed to identify alleles in Protein C (rs2069912), EPCR (rs2069948) and LNPEP (rs18059). As described in WO05/087789 (the '789 application), a SNP at position 4732 of the Protein C sequence shown in SEQ ID NO:1 of the '789 application (corresponding to rs2069912) and a SNP at position 4054 of the Epithelial Protein C receptor gene shown in SEQ ID NO:2 of the '789 application (corresponding to rs2069948) can be used as indicators of recovery or response to treatment in patients with SIRS. Similarly, WO07/085,087 describes the use of a leucyl/cystinyl aminopeptidase (LNPEP) SNP (rs18059) as a prognostic indicator of recovery or response to treatment. The entire content of WO05087789 and WO07085087 are incorporated herein by reference.

The primer sequences used in the assay were as follows:

| | |
|---|---|
| Protein C rs2069912 forward T allele primer: | |
| ACACTCATTTAACACTATTTCATT/iSp18/CCTCCTCTAGGATGCCTTTT | (SEQ ID NO: 1) |
| Protein C rs2069912 forward C allele primer: | |
| CTTTCTCATACTTTCAACTAATTT/iSp18/CCTCCTCTAGGATGCCTTTC | (SEQ ID NO: 2) |
| Protein C rs2069912 reverse primer (biotin labeled): | |
| /5Bio/ACAGACCTATCACCCAGGAA | (SEQ ID NO: 3) |
| EPCR rs2069948 forward T allele primer: | |
| CATAAATCTTCTCATTCTAACAAA/iSp18/CGGCCCAGGCTGAAGT | (SEQ ID NO: 4) |
| EPCR rs2069948 forward C allele primer: | |
| TACAACATCTCATTAACATATACA/iSp18/CGGCCCAGGCTGAAGC | (SEQ ID NO: 5) |
| EPCR rs2069948 reverse primer (biotin labeled): | |
| 5bio/GAAGTAGGAGATCTGGAGCAT | (SEQ ID NO: 6) |
| LNPEP rs18059 forward C allele primer: | |
| TTAATACAATTCTCTCTTTCTCTA/iSp18/TTGAGTGCTTTTACACACC | (SEQ ID NO: 7) |
| LNPEP rs18059 forward T allele primer: | |
| TCATCACTTTCTTTACTTTACATT/iSp18/ATTTGAGTGCTTTTACACACT | (SEQ ID NO: 8) |
| LNPEP rs18059 reverse primer (biotin labeled): | |
| /5Bio/CTATTCCGTCTCCTCGTTTATT | (SEQ ID NO: 9) |

PCR was performed in Starstedt Multiply—uStripPro 0.2 mL tubes at a final volume of 25 uL. Reagent concentrations were as follows: 3-12 pmol each primer; gDNA 100 ng; dNTPs (dATP, dCTP, dGTP and dTTP) each at 200 μM; 1U TaKaRa HotStart DNA polymerase; 1.5× TaKaRa buffer with MgCl₂ (15 mM Tris-HCl (pH 8.3), 75 mM KCl, 2.25 mM MgCl₂). Cycling conditions were as follows: 5 min at 95° C., 30 cycles [30 sec at 95° C., 30 sec at 60° C., 30 sec at 72° C.], then 2 min at 72° C., hold at 4° C.

Hybridization and detection was performed in a Costar Thermowell 96-well plate. 7.5 uL of the sepsis PCR product was added into 70 uL bead and reporter mix. The bead and reporter mix contained 1200 Luminex beads (coupled with specific anti-tag sequence) of each bead population selected for the assay, 1× buffer (0.2 M NaCl, 100 mM Tris, 0.08% Triton X-100, pH 8.0), and 1×10−6 g Invitrogen streptavidin-phycoerythrin (SA-PE) reporter. Incubation was at 37° C. for 30 min (protected from light). The plate was placed in the heat block of the Luminex instrument (temp at 37° C.) and 50 uL of the hyb/detection reaction was read. The results of the assay are represented in the graphs in FIGS. 6A-F.

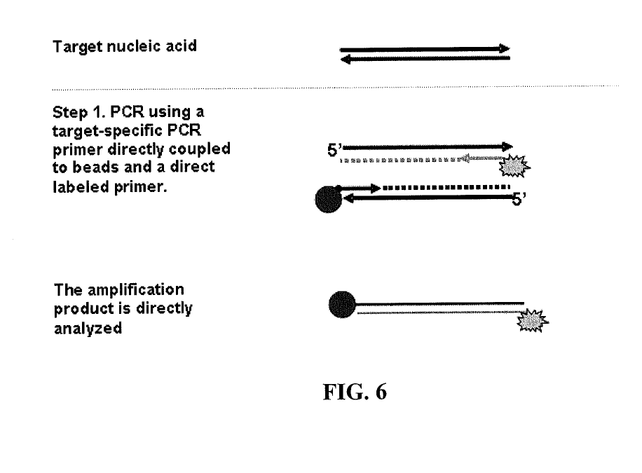

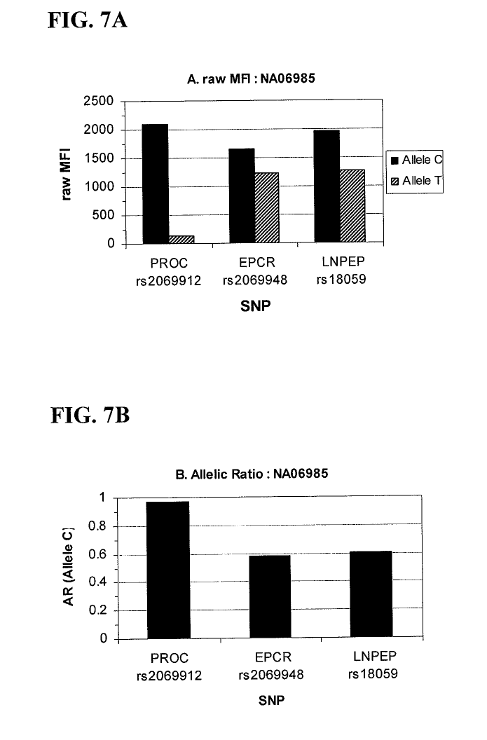

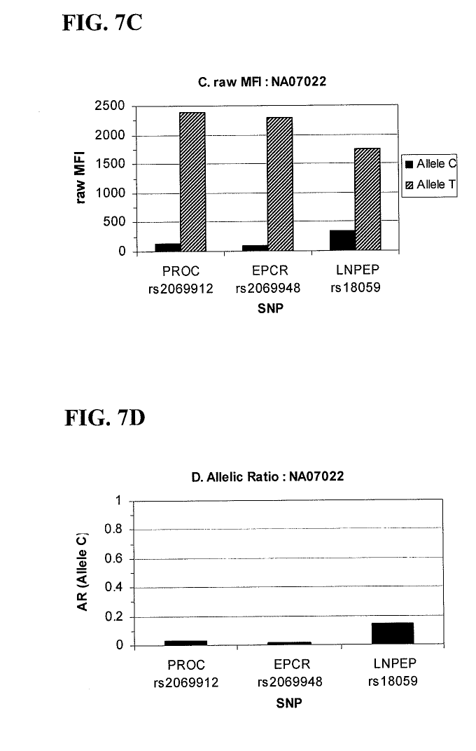

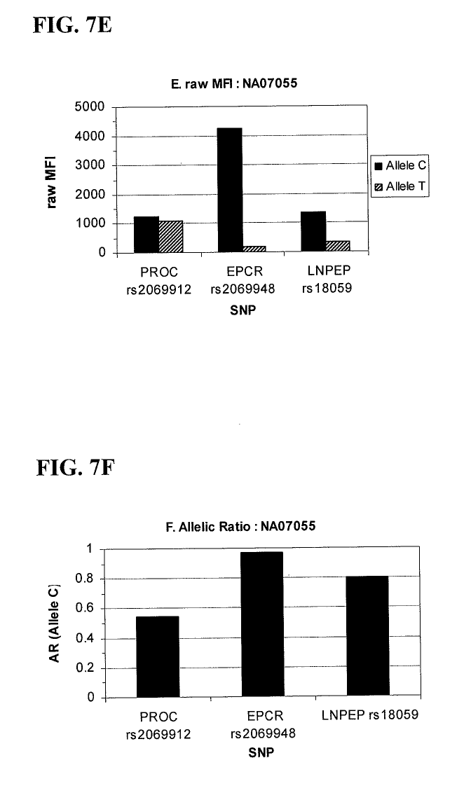

FIGS. 6A and 6B show the results of the 3-plex assay on sample NA06985, with the raw MFI shown in FIG. 7A and the allelic ratio shown in FIG. 7B. As shown by the allelic ratios, NA06985 is homozygous C/C at the rs2069912 polymorphic site of Protein C, heterozygous C/T for the rs2069948 polymorphic site of the EPCR, and heterozygous C/T for the rs18059 polymorphic site of LNPEP (FIG. 7B).

FIGS. 6C and 6D show the results of the 3-plex assay on sample NA07022, with the raw MFI shown in FIG. 7C and the allelic ratio shown in FIG. 7D. As shown by the allelic ratios, NA07022 is homozygous T/T at the rs2069912 polymorphic site of Protein C, homozygous T/T for the rs2069948 polymorphic site of the EPCR, and homozygous T/T for the rs18059 polymorphic site of LNPEP (FIG. 7D).

FIGS. 6E and 6F show the results of the 3-plex assay on sample NA07055, with the raw MFI shown in FIG. 7E and the allelic ratio shown in FIG. 7F. As shown by the allelic ratios, NA07055 is heterozygous C/T at the rs2069912 polymorphic site of Protein C, homozygous C/C for the rs2069948 polymorphic site of the EPCR, and homozygous C/C for the rs18059 polymorphic site of LNPEP (FIG. 7F).

### 2. Example 2

### One-Step Target Detection Assay—Singleplex Assay

Amplification and detection of a nucleic acid target (Coxsackie virus) was performed with a tagged and blocked target-specific PCR primer and a 5′-direct-labeled (HEX) target-specific PCR primer. Luminex beads coupled to anti-tag sequences were also included in the amplification reaction. Following the amplification reaction, detection of the labeled, double-stranded product was carried out on the Luminex instrument.

More specifically, the Coxsackie virus target sequence was amplified using in Starstedt Multiply uStripPro 0.2 mL tubes at a final volume of 15 uL. Reagent concentrations were as follows: 5 μmol each primer (HEX labeled); dNTPs (dATP, dCTP, dGTP and dTTP) each at 200 μM; 1.25× Qiagen OneStep RT-PCR buffer (Tris, KCl, (NH₄)₂SO₄ and DTT at non-disclosed concentrations), 3.125 mM MgCl₂, pH 8.7; 1.2 μL Qiagen OneStep RT-PCR Enzyme Mix (contains a non-disclosed combination of Omniscript and Sensiscript reverse transcriptases, and HotStar Taq polymerase); 1200 Luminex microspheres/beads (coupled with specific anti-tag sequence) of each bead population selected for the assay. Cycling conditions were: 50° C. for 30 min, 95° C. for 5 min, 30 cycles [30 sec at 95° C., 30 sec at 56° C., 30 sec at 72° C.], then 2 min at 72° C., hold at 4° C. The RT-PCR was carried out on samples from Zeptometrix (Coxsackie, Adenovirus, RSV A and Flu B) and a negative control (in which water was added to the RT-PCR instead of viral RNA or DNA).

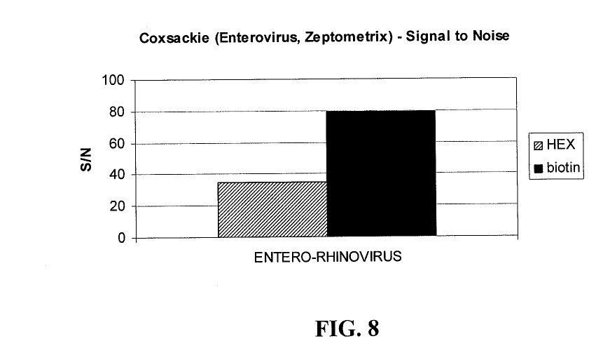

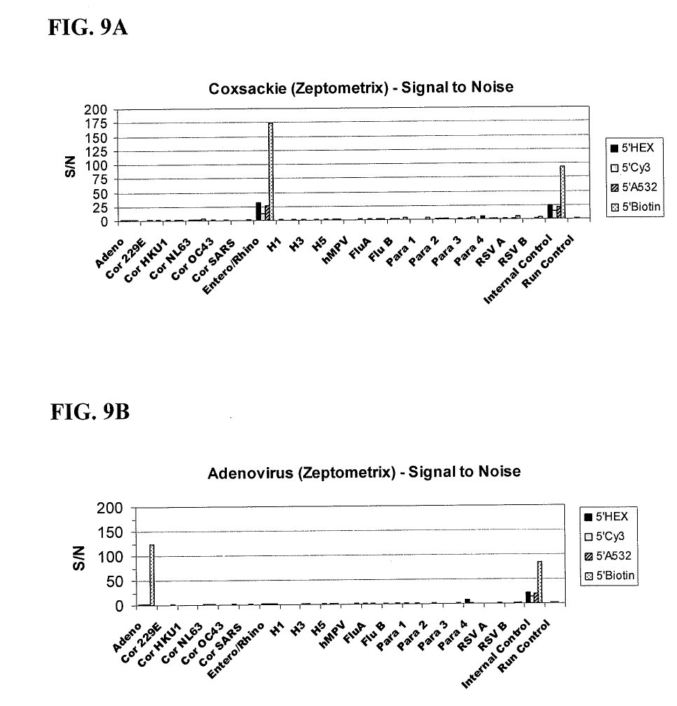

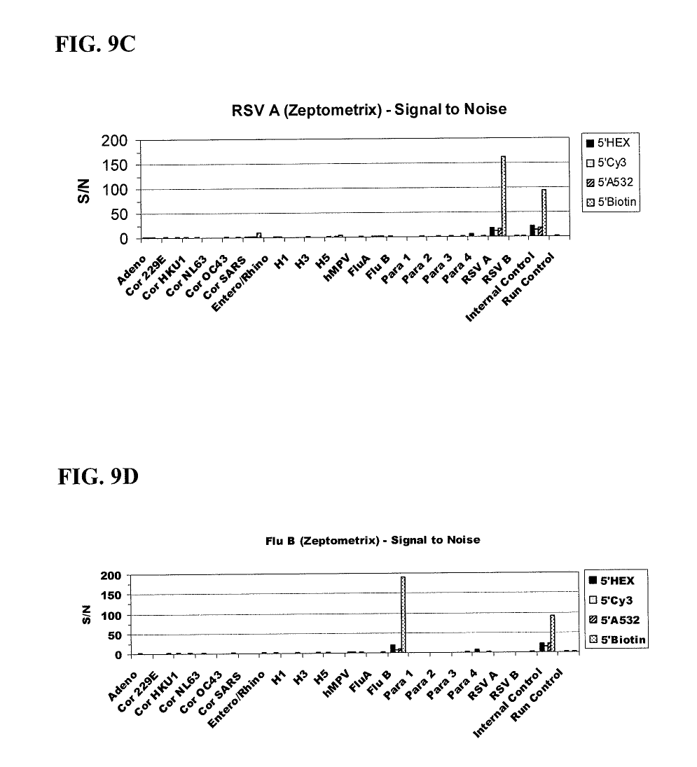

Hybridization and detection was performed in a Costar Thermowell 96-well plate. 60 uL of 1.25× buffer (0.25 M NaCl, 125 mM Tris, 0.1% v/v TritonX-100, pH 8.0) was added to 15 uL RVP (respiratory viral panel) RT-PCR product. The plate was placed in the heat block of the Luminex instrument (temp at 37° C.) and 50 uL of the hyb/detection reaction was read. The MFI (Median Fluorescence Intensity) for Coxsackie virus was 208. The MFI for the negative control was 6. Thus, the signal-to-noise ratio was 34.7 for the HEX-labeled one-step target detection assay (FIG. 8).

A two-step biotin-labeled assay was performed in parallel for comparison with the one-step assay. PCR was performed as described above except that biotin-labeled primers were used instead of HEX-labeled primers and there were no Luminex microspheres/beads (coupled with specific anti-tag sequence) present during the PCR reaction. Hybridization and detection was performed in a Costar Thermowell 96-well plate adding 2.5 uL of the sepsis PCR product into 70 uL bead and reporter mix. The bead and reporter mix contained 1200 Luminex microspheres/beads (coupled with specific anti-tag sequence) of each bead population selected for the assay, 1× buffer (0.2 M NaCl, 100 mM Tris, 0.08% Triton X-100, pH 8.0), and 1×10−6 g Invitrogen streptavidin-phycoerythrin (SA-PE) reporter. Incubation was at 37° C. for 30 min (protected from light). The plate was placed in the heat block of the Luminex instrument (temp at 37° C.) and 50 uL of the hyb/detection reaction was read. The MFI for Coxsackie virus was 3566. The MFI for the negative control was 45. Thus, the signal-to-noise ratio was 79.24 for the biotin-labeled two-step target detection assay (FIG. 8).

### 3. Example 3

### One-Step Target Detection Assay—Multiplex Assay

In this multiplexed one-step assay format, amplification and detection of 5 nucleic acid targets was performed with one PCR primer for each target that was tagged with a universal sequence and a blocker, and a second PCR primer for each target that was labeled with a reporter molecule (either HEX, Cy3 or Alexa532) that could be measured directly. Luminex beads coupled to anti-tag sequences were also included in the amplification reaction. Following the amplification reaction, detection of the labeled and tagged, partially double-stranded products was carried out on a Luminex instrument. A schematic representation of a one-step target detection assay is shown in FIG. 1.

More specifically, PCR primer pairs for 5 targets from a Respiratory Viral Panel (RVP) assay were selected. The 5 targets included Adeno, Flu B, Coxsackie virus (Enterovirus/Rhinovirus), RSV A and the internal control, MS2. Each of the primer pairs included two target-specific PCR primers. One primer contained a unique universal sequence (tag) on the 5′ end and a blocker molecule (iSp18) positioned between the tag and target specific regions of the primer. The other primer was 5′ labeled with a fluorophore (either HEX, Cy3 or Alexa532) or biotin. The biotin version of the assay, which served as the control format, was carried out as a 2-step assay.

RT-PCR was carried out on 4 viral controls/samples from Zeptometrix (Coxsackie, Adenovirus, RSV A and Flu B) and a negative control (in which water was added to the RT-PCR instead of viral RNA or DNA). MS2 control was added to each of the Zeptometrix samples prior to their extraction. Each 5-plex RT-PCR contained the 5 primer sets (one for each target) labeled with the same reporter (i.e. either HEX, Cy3, A532 or biotin), bead mix (which included 21 bead subpopulations), Qiagen OneStep RT-PCR buffer, dNTPs, and Qiagen OneStep Enzyme Mix. More specifically, the reaction was performed in Starstedt Multiply—uStripPro, 0.2 mL tubes at a final volume of 15 uL. Reagent concentrations were: 2-5 pmol each primer (for 1-step, fluor format) or 2 pmol each primer (for 2-step, biotin format); dNTPs (dATP, dCTP, dGTP and dTTP) each 200 μM; 1× Qiagen OneStep RT-PCR buffer (Tris, KCl, (NH₄)₂SO₄ and DTT at non-disclosed concentrations), 2.5 mM MgCl₂, pH 8.7; 1.2 μL Qiagen OneStep RT-PCR Enzyme Mix (contains a non-disclosed combination of Omniscript and Sensiscript reverse transcriptases, and HotStar Taq polymerase; 1200 Luminex microspheres/beads (coupled with specific anti-tag sequence) of each bead population selected for the assay. Cycling conditions were: 50° C. for 30 min, 95° C. for 5 min, 34 cycles [30 sec at 95° C., 30 sec at 56° C., 30 sec at 72° C.], then 2 min at 72° C., hold at 4° C.

Hybridization and detection was performed in a Costar Thermowell 96-well plate. For the fluor-labeled amplification products (1-step format), 5 uL of RT-PCR products were combined with 70 uL of 1×BN buffer (0.2 M NaCl, 100 mM Tris, 0.08% Triton X-100, pH 8.0). The plate was placed in the heat block of the Luminex instrument (temp at 37° C.) and 50 uL of the hyb/detection reaction was read. Since the streptavidin-phycoerythrin reporter used in the biotin assay denatures at the high temperatures reached during PCR cycling, detection of the biotin-labeled PCR product using this reporter was carried out in a separate step following PCR and then analyzed on the Luminex instrument.

The results of assay are represented in the graphs in FIGS. 8A-D. The x-axis for each graph represents the bead subpopulation used in the assay (each bead subpopulation is named for the RVP target that it detects). The y-axis represents the calculated signal-to-noise ratio, where the noise is the raw MFI (median fluorescence intensity) of the given bead subpopulation/target for the negative control sample. In this study all background signals were <80 MFI. Each graph in FIGS. 8A-D compares the HEX, Cy3, Alexa532 and biotin assay formats for the specific sample tested.

Three out of the 4 viral targets tested (RSV A, Flu B and Coxsackie) were detected using the one-step assay format. As expected, the sensitivity (based on signal-to-noise calculations) was superior with the 2-step assay using the streptavidin-phycoerythrin reporter (vs the 1-step format). This decreased sensitivity may explain why the Adenovirus sample was not detected by the 1-step assay (using any of the 3 tested fluor labels) but was detected by the 2-step assay. Comparing the 3 fluorophores tested in the one-step assay, HEX appeared to provide the best signal to noise values (A532 gave similar but slightly lower S/N). These results suggest that the assay may be further optimized to improve performance such that sensitivity of the 1-step assay format is comparably to that of the 2-step assay format.

This study demonstrated the feasibility of multiplexing with the described one-step technology. Since the assay was performed in one step, it significantly reduces assay complexity, hands-on time, turn-around-time, and risk of PCR product contamination.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,284,412
U.S. Pat. No. 4,498,766
U.S. Pat. No. 4,659,774
U.S. Pat. No. 4,661,913
U.S. Pat. No. 4,682,195
U.S. Pat. No. 4,683,202
U.S. Pat. No. 4,714,682
U.S. Pat. No. 4,767,206
U.S. Pat. No. 4,774,189
U.S. Pat. No. 4,816,571
U.S. Pat. No. 4,857,451
U.S. Pat. No. 4,883,750
U.S. Pat. No. 4,942,124
U.S. Pat. No. 4,959,463
U.S. Pat. No. 4,989,977
U.S. Pat. No. 5,137,806
U.S. Pat. No. 5,141,813
U.S. Pat. No. 5,160,974
U.S. Pat. No. 5,264,566
U.S. Pat. No. 5,428,148
U.S. Pat. No. 5,478,722
U.S. Pat. No. 5,525,494
U.S. Pat. No. 5,539,082
U.S. Pat. No. 5,554,744
U.S. Pat. No. 5,574,146
U.S. Pat. No. 5,595,890
U.S. Pat. No. 5,602,244
U.S. Pat. No. 5,639,611
U.S. Pat. No. 5,645,897
U.S. Pat. No. 5,654,413
U.S. Pat. No. 5,705,629
U.S. Pat. No. 5,714,331
U.S. Pat. No. 5,719,262
U.S. Pat. No. 5,736,330
U.S. Pat. No. 5,736,336
U.S. Pat. No. 5,766,855
U.S. Pat. No. 5,773,571
U.S. Pat. No. 5,786,461
U.S. Pat. No. 5,837,832
U.S. Pat. No. 5,837,860
U.S. Pat. No. 5,840,873
U.S. Pat. No. 5,843,640
U.S. Pat. No. 5,843,650
U.S. Pat. No. 5,843,651
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,487
U.S. Pat. No. 5,849,497
U.S. Pat. No. 5,849,546
U.S. Pat. No. 5,849,547
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,882,864
U.S. Pat. No. 5,891,625
U.S. Pat. No. 5,905,024
U.S. Pat. No. 5,908,845
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 5,981,180
U.S. Pat. No. 6,057,107
U.S. Pat. No. 6,103,463
U.S. Pat. No. 6,287,778
U.S. Pat. No. 6,322,971
U.S. Pat. No. 7,226,737
U.S. Pat. No. 7,226,737
U.S. Pub. Appln. 2005/0191625
Egholm et al., Nature, 365(6446):566-568, 1993.
EP Appln. 266,032
EP Appln. 320,308
EP Appln. 329,822
Fodor et al., Biochemistry, 30(33):8102-8108, 1991.
Froehler et al., Nucleic Acids Res., 14(13):5399-5407, 1986.
Frohman, In: PCR Protocols. A Guide To Methods And Applications, Academic Press, N.Y., 1990.
GB Appln. 2 202 328
Holmstrom et al., Anal. Biochem. 209:278-283, 1993.
Koshkin and Dunford, J. Biol. Chem., 273(11):6046-6049, 1998a.
Koshkin and Wengel, J. Org. Chem., 63(8):2778-2781, 1998b.
Kwoh et al., Proc. Natl. Acad. Sci. USA, 86:1173, 1989.
Mueller et al., Current Protocols in Mol. Biol.; 15:5, 1993.
Newton et al., Nucl. Acids Res. 21:1155-1162, 1993.
Ohara et al., Proc. Natl. Acad. Sci. USA, 86:5673-5677, 1989.
PCT Appln. WO 00/47766
PCT Appln. WO 88/10315
PCT Appln. WO 89/06700
PCT Appln. WO 90/07641
PCT Appln. WO 92/20702
PCT Appln. WO 93/17126
PCT Appln. WO 9731256
PCT Appln. WO05087789
PCT Appln. WO07/085,087
PCT Appln. PCT/EP/01219
PCT Appln. PCT/US87/00880
PCT Appln. PCT/US89/01025
Pease et al., Proc. Natl. Acad. Sci. USA, 91:5022-5026, 1994.
Rasmussen et al., Anal. Biochem, 198:138-142, 1991.
Running et al., BioTechniques 8:276-277, 1990.
Santalucia et al., Biochemistry; 38:3468-3477, 1999.
Wahlestedt et al., Proc. Natl. Acad. Sci. USA, 97(10):5633-5638, 2000.
Walker et al., Nucleic Acids Res. 20(7):1691-1696, 1992.

1. A method for amplifying a target nucleic acid sequence comprising:
(a) providing a first primer pair comprising:
a first primer comprising a target specific sequence, a tag sequence 5′ of the target specific sequence, and a blocker between the target specific sequence and the tag sequence,
a second primer comprising a target specific sequence;
(b) a reporter attached to either the second primer or to a dNTP;
(c) providing a capture complex comprising an anti-tag sequence attached to a solid support; and
(d) combining the first primer pair, the reporter, the capture complex, and a sample comprising a target nucleic acid sequence under conditions suitable for amplification of the target nucleic acid sequence and hybridization of the amplified target nucleic acid sequence to the capture complex.

2. The method of claim 1, wherein the blocker is an iSp18 moiety.

3. (canceled)

4. The method of claim 1, wherein the reporter is attached to the second primer.

5. The method of claim 1, wherein the reporter is attached to the dNTP.

6. The method of claim 1, wherein the reporter is biotin.

7-8. (canceled)

9. The method of claim 1, wherein the solid support is a bead.

10-15. (canceled)

16. The method of claim 1, further comprising detecting the amplified target nucleic acid sequence.

17-18. (canceled)

19. The method of claim 1, wherein the sample comprises at least a second target nucleic acid sequence.

20. The method of claim 19, wherein at least a second primer pair is combined with the first primer pair, the reporter, the capture complex, and the sample comprising the target nucleic acid sequences under conditions suitable for amplification of the target nucleic acid sequences and hybridization of the amplified target nucleic acid sequences to the capture complex.

21. The method of claim 20, wherein the first primer pair and the second primer pair prime the amplification of two different target nucleic acid sequences in the sample.

22. The method of claim 21, wherein the different amplified target nucleic acid sequences hybridize to distinguishable capture complexes.

23. (canceled)

24. The method of claim 23, wherein the capture complexes are optically distinguishable.

25-36. (canceled)

37. A method for amplifying a target nucleic acid sequence comprising:
(a) providing a first primer pair comprising:
a first primer comprising a target specific sequence, wherein the first primer is covalently coupled to a solid support,
a second primer comprising a target specific sequence;
(b) a reporter attached to either the second primer or to a dNTP;
(c) combining the first primer pair and the reporter with a sample comprising a target nucleic acid sequence under conditions suitable for amplification of the target nucleic acid sequence, wherein the target nucleic acid sequence is amplified.

38. The method of claim 37, wherein the reporter is biotin.

39-40. (canceled)

41. The method of claim 37, wherein the solid support is a bead.

42-47. (canceled)

48. The method of claim 37, further comprising detecting the amplified target nucleic acid sequence.

49-50. (canceled)

51. The method of claim 37, wherein the sample comprises at least a second target nucleic acid sequence.

52. The method of claim 51, wherein at least a second primer pair is combined with the first primer pair and the sample comprising the target nucleic acid sequences under conditions suitable for amplification of the target nucleic acid sequences.

53. The method of claim 52, wherein the first primer pair and the second primer pair prime the amplification of two different target nucleic acid sequences in the sample.

54. The method of claim 53, wherein the different amplified target nucleic acid sequences are coupled to distinguishable solid supports.

55. (canceled)

56. The method of claim 55, wherein the solid supports are optically distinguishable.

57-66. (canceled)


SEQUENCE LISTING (9 sequences)

SEQ ID NO: 1 - DNA, 44 bp, Artificial
  Synthetic primer
acactcatttaacactatttcattcctcctctaggatgcctttt

SEQ ID NO: 2 - DNA, 44 bp, Artificial
  Synthetic primer
ctttctcatactttcaactaatttcctcctctaggatgcctttc

SEQ ID NO: 3 - DNA, 20 bp, Artificial
  Synthetic primer
acagacctatcacccaggaa

SEQ ID NO: 4 - DNA, 40 bp, Artificial
  Synthetic primer
cataaatcttctcattctaacaaacggcccaggctgaagt

SEQ ID NO: 5 - DNA, 40 bp, Artificial
  Synthetic primer
tacaacatctcattaacatatacacggcccaggctgaagc

SEQ ID NO: 6 - DNA, 21 bp, Artificial
  Synthetic primer
gaagtaggagatctggagcat

SEQ ID NO: 7 - DNA, 43 bp, Artificial
  Synthetic primer
ttaatacaattctctctttctctattgagtgcttttacacacc

SEQ ID NO: 8 - DNA, 45 bp, Artificial
  Synthetic primer
tcatcactttctttactttacattatttgagtgcttttacacact

SEQ ID NO: 9 - DNA, 22 bp, Artificial
  Synthetic primer
ctattccgtctcctcgtttatt